(19) United States
(12) Patent Application Publication
(10) Pub. No.: US 2016/0046651 A1
(43) Pub. Date: Feb. 18, 2016

(54) TREATMENT OF MALARIA USING INHIBITORS OF THE ISPD ENZYME IN THE NON-MEVALONATE PATHWAY

(71) Applicants: Washington University, St. Louis, MO (US); University of Liverpool, Liverpool (GB)

(72) Inventors: Audrey Odom, St. Louis, MO (US); Paul O'Neill, Liverpool (GB); Neil Berry, Liverpool (GB)

(21) Appl. No.: 14/804,440

(22) Filed: Jul. 21, 2015

Related U.S. Application Data
(60) Provisional application No. 62/027,642, filed on Jul. 22, 2014.

(51) Int. Cl. C07D 513/04; C07D 417/04; C07D 417/10; C07D 275/04; C07D 417/12

(52) U.S. Cl. CPC C07D 513/04; C07D 275/04; C07D 417/12; C07D 417/10; C07D 417/04

(57) ABSTRACT

Compounds are disclosed that inhibit the methylerythritol cytidyltransferase (IspD) enzyme in the non-mevalonate pathway (MEP pathway), which is present in many organisms including the P. falciparum parasite. Inhibitors of the IspD enzyme in the non-mevalonate pathway of the P. falciparum parasite are useful for treating malaria.

### CROSS-REFERENCE TO RELATED APPLICATIONS

This application claims benefit of U.S. provisional application Ser. No. 62/027,642, filed Jul. 22, 2014, the entire disclosure of which is incorporated herein by reference.

### FIELD OF THE INVENTION

The present invention generally relates to compounds that inhibit the methylerythritol cytidyltransferase (IspD) enzyme in the non-mevalonate pathway (MEP pathway), which is present in many organisms including the P. falciparum parasite. Inhibitors of the IspD enzyme in the non-mevalonate pathway of the P. falciparum parasite are useful for treating malaria. Accordingly, the present invention also relates to methods of treating malaria by administering a composition comprising an IspD enzyme inhibitor compound.

### BACKGROUND OF THE INVENTION

Malaria remains a major threat to global health, with over 250 million cases per year and one million deaths per year, primarily in children under the age of five. The primary parasites that cause malaria, P. falciparum and P. vivax, are largely resistant to older therapies. Quinine and derivatives such as chloroquine have been used for decades for the treatment of uncomplicated malaria, and are often the drugs of last resort for the treatment of severe malaria. However, the usefulness of these drugs has rapidly declined in parts of the world where resistant strains of P. falciparum and P. vivax have emerged and are now widespread. P. falciparum is also increasingly resistant to relatively newer frontline agents that have been developed (e.g., semi-synthetic artemesinins). Therefore, an urgent need remains for the discovery of new antimalarial agents.

Isoprenoids represent a diverse family of over 35,000 natural products, including sterols and terpenes. The biosynthesis of isoprenoids occurs through the repeated condensation of a key precursor, isopentenyl pyrophosphate (IPP). Mammals and fungi derive IPP from a coenzyme A (CoA)-dependent pathway, which proceeds through the key intermediate mevalonate. Recent studies have identified the MEP pathway (also known as the non-mevalonate and the 1-deoxy-d-xylulose 5-phosphate (DOXP) pathway) as an alternative biosynthetic route to IPP. The MEP pathway is utilized by plants, algae, bacteria and protozoa, but is crucially absent in mammalian systems, which instead utilize the mevalonate pathway to synthesize IPP. MEP pathway enzymes are known to be present in all intraerythrocytic stages of the P. falciparum parasite.

Accordingly, the MEP pathway in parasites including P. falciparum is an attractive target for next generation antimalarial therapies. Thus, there remains a need for compounds that are capable of disrupting this critical pathway to treat malaria.

### SUMMARY OF THE INVENTION

Briefly, the present invention relates to compounds that inhibit the methylerythritol cytidyltransferase (IspD) enzyme in the non-mevalonate pathway (MEP pathway). The present invention also relates to methods of treating malaria by administering a composition comprising an IspD enzyme inhibitor compound.

IspD enzyme inhibitor compounds of the present invention include compounds of Formula I

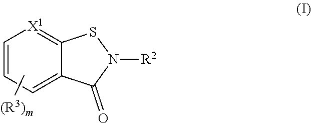

where

X¹ is C—R¹ or N;

R¹ is hydrogen, halo, hydroxy, C₁-C₄ alkyl, C₁-C₄ alkoxy, halo-substituted C₁-C₄ alkyl, or amino;

R² is:

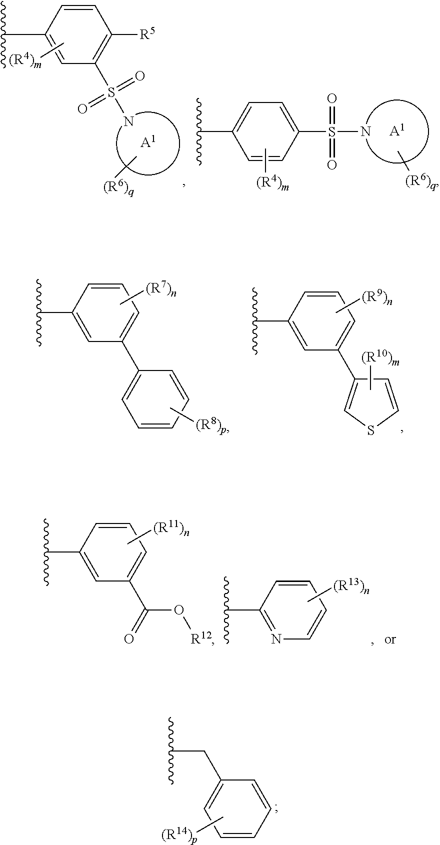

each R³ is independently halo, hydroxy, C₁-C₄ alkyl, C₁-C₄ alkoxy, halo-substituted C₁-C₄ alkyl, or amino;

each R⁴ is halo, hydroxy, C₁-C₄ alkyl, C₁-C₄ alkoxy, halo-substituted C₁-C₄ alkyl, amino, or a nitrogen-containing aliphatic ring;

R⁵ is hydrogen, halo, hydroxy, C₁-C₄ alkyl, C₁-C₄ alkoxy, halo-substituted C₁-C₄ alkyl, or amino;

each R⁶ is independently halo, hydroxy, C₁-C₄ alkyl, C₁-C₄ alkoxy, halo-substituted C₁-C₄ alkyl, or amino;

each R⁷ is independently halo, hydroxy, C₁-C₄ alkyl, C₁-C₄ alkoxy, halo-substituted C₁-C₄ alkyl, or amino;

each R⁸ is independently halo, hydroxy, C₁-C₄ alkyl, C₁-C₄ alkoxy, halo-substituted C₁-C₄ alkyl, or amino;

each R⁹ is independently halo, hydroxy, C₁-C₄ alkyl, C₁-C₄ alkoxy, halo-substituted C₁-C₄ alkyl, or amino;

each R¹⁹ is independently halo, hydroxy, C₁-C₄ alkyl, C₁-C₄ alkoxy, halo-substituted C₁-C₄ alkyl, or amino;

each R¹⁰ is halo, hydroxy, C₁-C₄ alkyl, C₁-C₄ alkoxy, halo-substituted C₁-C₄ alkyl, or amino;

R¹² is hydrogen or C₁-C₄ alkyl;

each R¹³ is independently halo, hydroxy, C₁-C₄ alkyl, C₁-C₄ alkoxy, halo-substituted C₁-C₄ alkyl, or amino;

each R¹⁴ is independently halo, hydroxy, C₁-C₄ alkyl, C₁-C₄ alkoxy, halo-substituted C₁-C₄ alkyl, or amino;

each A¹ is an aliphatic heterocyclic ring;

each m is independently 0 to 3;

each n is independently 0 to 4;

each p is independently 0 to 5; and

q is 0 to 10.

IspD enzyme inhibitor compounds of the present invention also include compounds of Formula II

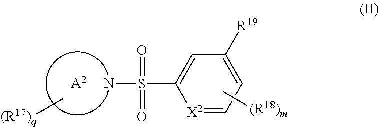

where

X² is C—R¹⁶, or N;

R¹⁶ is hydrogen, halo, hydroxy, C₁-C₄ alkyl, C₁-C₄ alkoxy, halo-substituted C₁-C₄ alkyl, or amino;

each R¹⁷ is independently halo, hydroxy, C₁-C₄ alkyl, C₁-C₄ alkoxy, halo-substituted C₁-C₄ alkyl, or amino;

each R¹⁸ is independently halo, hydroxy, C₁-C₄ alkyl, C₁-C₄ alkoxy, halo-substituted C₁-C₄ alkyl, or amino;

R¹⁹ is

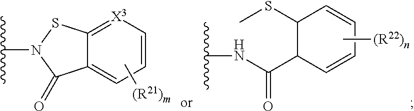

X³ is C—R²⁰ or N;

R²⁰ is hydrogen, halo, hydroxy, C₁-C₄ alkyl, C₁-C₄ alkoxy, halo-substituted C₁-C₄ alkyl, or amino;

each R²¹ is independently halo, hydroxy, C₁-C₄ alkyl, C₁-C₄ alkoxy, halo-substituted C₁-C₄ alkyl, or amino;

each R²² is independently halo, hydroxy, C₁-C₄ alkyl, C₁-C₄ alkoxy, halo-substituted C₁-C₄ alkyl, or amino;

A² is an aliphatic heterocyclic ring;

each m is independently 0 to 3;

n is 0 to 4; and

q is 0 to 10.

The present invention includes methods of treating a disease caused by an organism possessing the MEP pathway (e.g., malaria) in a subject in need thereof. The method comprises administering a pharmaceutical composition comprising a therapeutically effective amount of a compound of Formula I, II, and combinations thereof.

The present invention further includes a method of treating a disease caused by an organism possessing the MEP pathway (e.g., malaria) in a subject in need thereof comprising administering a pharmaceutical composition comprising a therapeutically effective amount of a compound selected from the group consisting of

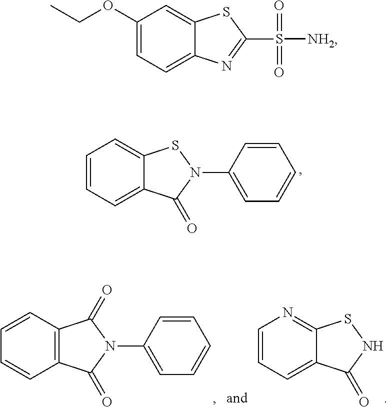 [SMILES: CCOc1ccc2nc(S(N)(=O)=O)sc2c1 ; O=c1c2ccccc2sn1-c1ccccc1 ; O=C1c2ccccc2C(=O)N1c1ccccc1 ; O=c1[nH]sc2ncccc12]

Other objects and features will be in part apparent and in part pointed out hereinafter.

### BRIEF DESCRIPTION OF THE DRAWINGS

FIG. 1 shows a schematic of isoprenoid biosynthesis via the MEP pathway.

FIG. 2 shows a comparison of in vitro activity against PfIspF (IC₅₀) and cellular activity against cultured malaria parasites (EC₅₀).

FIG. 3 presents a summary of mass spectrometry data to determine the concentrations of MEP pathway metabolites with and without treatment with IspD enzyme inhibitor compound 41.

### DESCRIPTION OF THE PREFERRED EMBODIMENTS

Generally, the present invention is directed to compounds that inhibit the IspD enzyme in the MEP pathway and methods of treating diseases caused by parasitic organisms that possess the MEP pathway. The MEP pathway is present in many organisms including the P. falciparum parasite. Accordingly, inhibitors of the IspD enzyme in the MEP pathway of the P. falciparum parasite are useful for treating malaria.

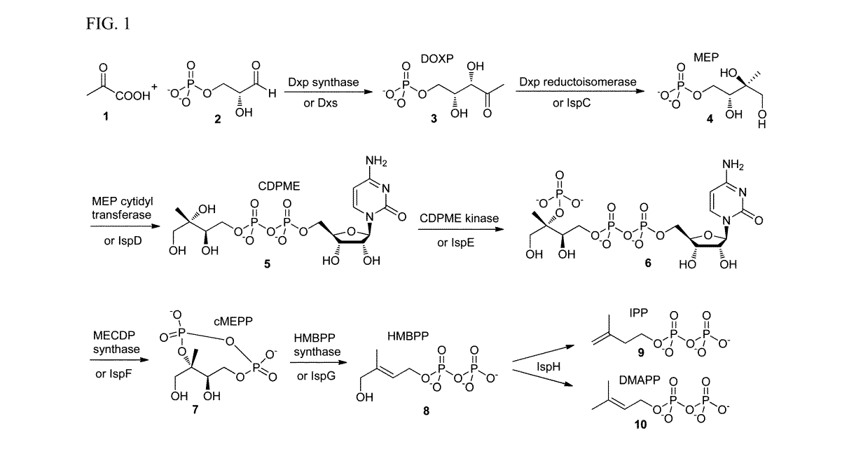

A schematic of isoprenoid biosynthesis via the MEP pathway is shown in FIG. 1. The MEP pathway is catalyzed by nine enzymes over a total of eight steps. The enzymes in this pathway are named according to E. coli nomenclature, although there is a proposed standardized nomenclature system in the literature (1). The pathway begins with the condensation of pyruvate 1 and glyceraldehyde 3-phosphate (GAP) 2, catalyzed by 1-deoxy-d-xylulose-5-phosphate synthase (Dxs), with thiamine pyrophosphate (TPP) acting as a cofactor. Pyruvate is thought to form a covalent intermediate with TPP, allowing reaction with glyceraldehyde 3-phosphate. The second enzyme in the pathway, 1-deoxy-d-xylulose-5-phosphate reductoisomerase (IspC), catalyzes both an intramolecular isomerization and reduction to generate 2C-methyl-d-erythritol 4-phosphate 4 (MEP). Fosmidomycin, a potent inhibitor of the MEP pathway, inhibits this enzyme from multiple organisms (2). In the next stage, MEP is coupled to cytidine triphosphate to produce 4-diphosphocytidyl-2C-methyl-d-erythritol (CDP-ME) 5 by 4-diphosphocytidyl-2C-methyl-d-erythritol cytidyltransferase (IspD). CDP-ME then undergoes phosphorylation by an ATP dependent kinase, 4-diphosphocytidyl-2C-methyl-d-erythritol kinase (IspE). 2C-Methyl-d-erythritol-2, 4-cyclodiphosphate synthase (IspF) then catalyzes the cyclization of 4-diphospho-cytidyl-2C-methyl-d-erythritol 2-phosphate 6 into 4-diphospho-cytidyl-2C-methyl-d-erythritol 2,4-cyclodiphosphate 7 (cMEPP). A two electron reduction of cMEPP forms 2-methyl-2-(E)-butenyl diphosphate 8, followed by conversion to IPP 9 and DMAPP 10. These steps are catalyzed by 1-hydroxy-2-methyl-2-(E)-butenyl-4-diphosphate synthase (IspG) and 4-hydroxy-3-methyl-2-(E)-butenyl-4-diphosphate reductase (IspH), respectively.

Isoprenoid biosynthesis via the MEP pathway has been shown to be essential to development in many organisms (3-7). Compounds that inhibit the IspD protein halt further development, which kills the organism. Thus, inhibitors of IspD are capable of killing parasites such as malaria that possess the MEP pathway. MEP pathway enzymes are present in all intraerythrocytic stages of P. falciparum. A plastid organelle known as the apicoplast, present within Plasmodium spp parasites, has recently been shown to serve a single function, namely the biosynthesis of isoprenoid precursors during blood stage growth, additionally validating the MEP pathway as a viable drug target (9). As drug resistance to frontline treatments for malaria emerges for conventional frontline treatments, the compounds of the present invention advantageously provide inhibition of a new therapeutic target, the IspD enzyme.

In one aspect, the IspD inhibitors of the present invention include compounds of Formula I

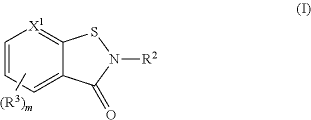

where

X¹ is C—R¹ or N;

R¹ is hydrogen, halo, hydroxy, C₁-C₄ alkyl, C₁-C₄ alkoxy, halo-substituted C₁-C₄ alkyl, or amino;

R² is:

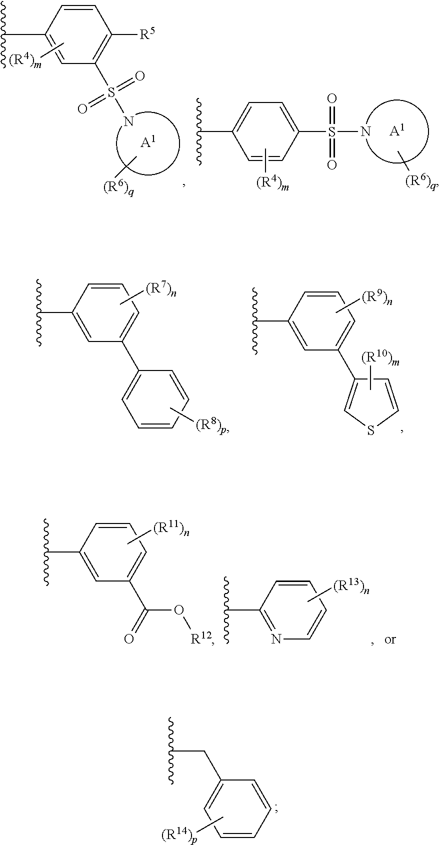

each R³ is independently halo, hydroxy, C₁-C₄ alkyl, C₁-C₄ alkoxy, halo-substituted C₁-C₄ alkyl, or amino;

each R⁴ is halo, hydroxy, C₁-C₄ alkyl, C₁-C₄ alkoxy, halo-substituted C₁-C₄ alkyl, amino, or a nitrogen-containing aliphatic ring;

R⁵ is hydrogen, halo, hydroxy, C₁-C₄ alkyl, C₁-C₄ alkoxy, halo-substituted C₁-C₄ alkyl, or amino;

each R⁶ is independently halo, hydroxy, C₁-C₄ alkyl, C₁-C₄ alkoxy, halo-substituted C₁-C₄ alkyl, or amino;

each R⁷ is independently halo, hydroxy, C₁-C₄ alkyl, C₁-C₄ alkoxy, halo-substituted C₁-C₄ alkyl, or amino;

each R⁸ is independently halo, hydroxy, C₁-C₄ alkyl, C₁-C₄ alkoxy, halo-substituted C₁-C₄ alkyl, or amino;

each R⁹ is independently halo, hydroxy, C₁-C₄ alkyl, C₁-C₄ alkoxy, halo-substituted C₁-C₄ alkyl, or amino;

each R¹⁰ is independently halo, hydroxy, C₁-C₄ alkyl, C₁-C₄ alkoxy, halo-substituted C₁-C₄ alkyl, or amino;

each R¹¹ is halo, hydroxy, C₁-C₄ alkyl, C₁-C₄ alkoxy, halo-substituted C₁-C₄ alkyl, or amino;

R¹² is hydrogen or C₁-C₄ alkyl;

each R¹³ is independently halo, hydroxy, C₁-C₄ alkyl, C₁-C₄ alkoxy, halo-substituted C₁-C₄ alkyl, or amino;

each R¹⁴ is independently halo, hydroxy, C₁-C₄ alkyl, C₁-C₄ alkoxy, halo-substituted C₁-C₄ alkyl, or amino;

each A¹ is an aliphatic heterocyclic ring;

each m is independently 0 to 3;

each n is independently 0 to 4;

each p is independently 0 to 5; and

q is 0 to 10.

In various embodiments, X¹ is C—R¹ and R¹ is hydrogen, halo, hydroxy, C₁-C₄ alkyl, or C₁-C₄ alkoxy. In some embodiments, R¹ is hydrogen. In other embodiments, X¹ is N.

In various embodiments, the compounds of Formula I are characterized by one or more of the following:

R² is

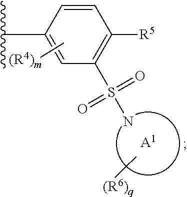

R⁴ is hydroxy, C₁-C₄ alkyl, C₁-C₄ alkoxy (e.g., methoxy or ethoxy), or a 5 to 7-membered nitrogen-containing aliphatic ring (e.g., piperydinyl or pyrrolidinyl);

R⁵ is hydrogen, hydroxy, C₁-C₄ alkyl (e.g., methyl or ethyl), or C₁-C₄ alkoxy (e.g., methoxy or ethoxy);

R⁶ is halo (e.g., F or Cl), C₁-C₄ alkyl (e.g., methyl or ethyl), C₁-C₄ alkoxy (e.g., methoxy or ethoxy), or amino (i.e., —NH₂);

A¹ is a 6 or 7-membered aliphatic heterocyclic ring optionally containing one or more additional heteroatoms selected from the group consisting of oxygen, nitrogen and combinations thereof;

m is 0 to 1; and

q is 0 to 1.

In the Formulas disclosed herein, when any of m, n, p, and q are 0, hydrogen is assumed to be present where appropriate.

In certain embodiments, the IspD inhibitor of Formula I is selected from the group consisting of:

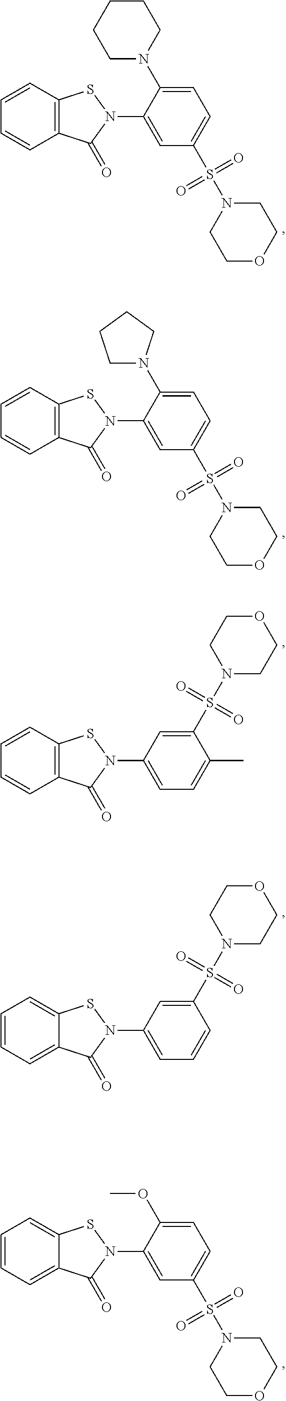 [SMILES: O=c1c2ccccc2sn1-c1cc(S(=O)(=O)N2CCOCC2)ccc1N1CCCCC1 ; O=c1c2ccccc2sn1-c1cc(S(=O)(=O)N2CCOCC2)ccc1N1CCCC1 ; Cc1ccc(-n2sc3ccccc3c2=O)cc1S(=O)(=O)N1CCOCC1 ; O=c1c2ccccc2sn1-c1cccc(S(=O)(=O)N2CCOCC2)c1 ; COc1ccc(S(=O)(=O)N2CCOCC2)cc1-n1sc2ccccc2c1=O]

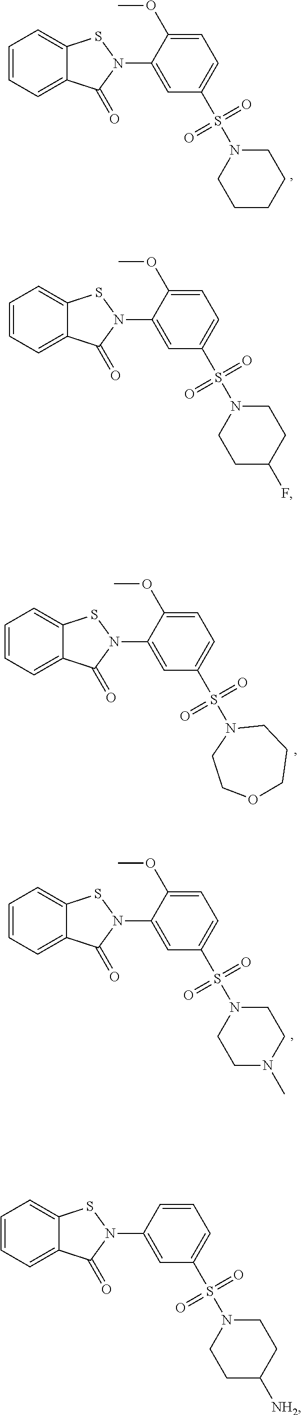 [SMILES: COc1ccc(S(=O)(=O)N2CCCCC2)cc1-n1sc2ccccc2c1=O ; COc1ccc(S(=O)(=O)N2CCC(F)CC2)cc1-n1sc2ccccc2c1=O ; COc1ccc(S(=O)(=O)N2CCCOCC2)cc1-n1sc2ccccc2c1=O ; COc1ccc(S(=O)(=O)N2CCN(C)CC2)cc1-n1sc2ccccc2c1=O ; NC1CCN(S(=O)(=O)c2cccc(-n3sc4ccccc4c3=O)c2)CC1]

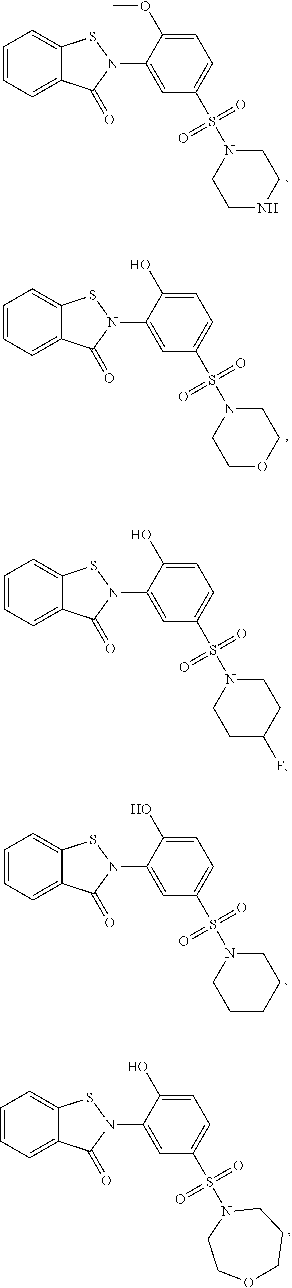 [SMILES: COc1ccc(S(=O)(=O)N2CCNCC2)cc1-n1sc2ccccc2c1=O ; O=c1c2ccccc2sn1-c1cc(S(=O)(=O)N2CCOCC2)ccc1O ; O=c1c2ccccc2sn1-c1cc(S(=O)(=O)N2CCC(F)CC2)ccc1O ; O=c1c2ccccc2sn1-c1cc(S(=O)(=O)N2CCCCC2)ccc1O ; O=c1c2ccccc2sn1-c1cc(S(=O)(=O)N2CCCOCC2)ccc1O]

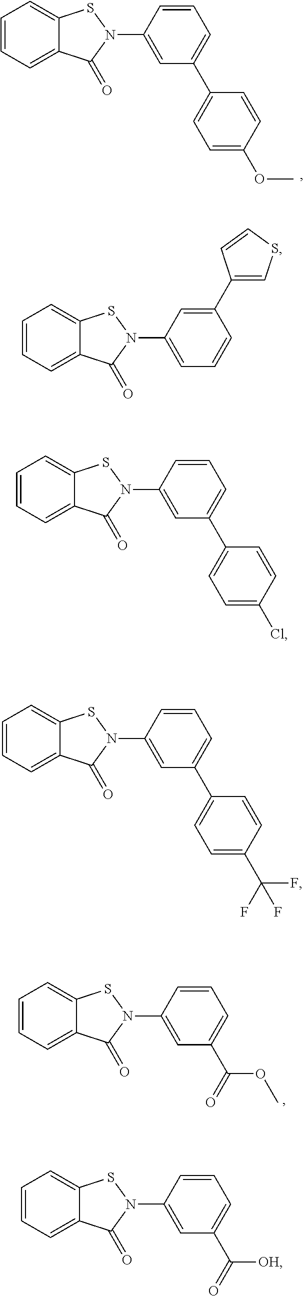 [SMILES: COc1ccc(-c2cccc(-n3sc4ccccc4c3=O)c2)cc1 ; O=c1c2ccccc2sn1-c1cccc(-c2ccsc2)c1 ; O=c1c2ccccc2sn1-c1cccc(-c2ccc(Cl)cc2)c1 ; O=c1c2ccccc2sn1-c1cccc(-c2ccc(C(F)(F)F)cc2)c1 ; COC(=O)c1cccc(-n2sc3ccccc3c2=O)c1 ; O=C(O)c1cccc(-n2sc3ccccc3c2=O)c1]

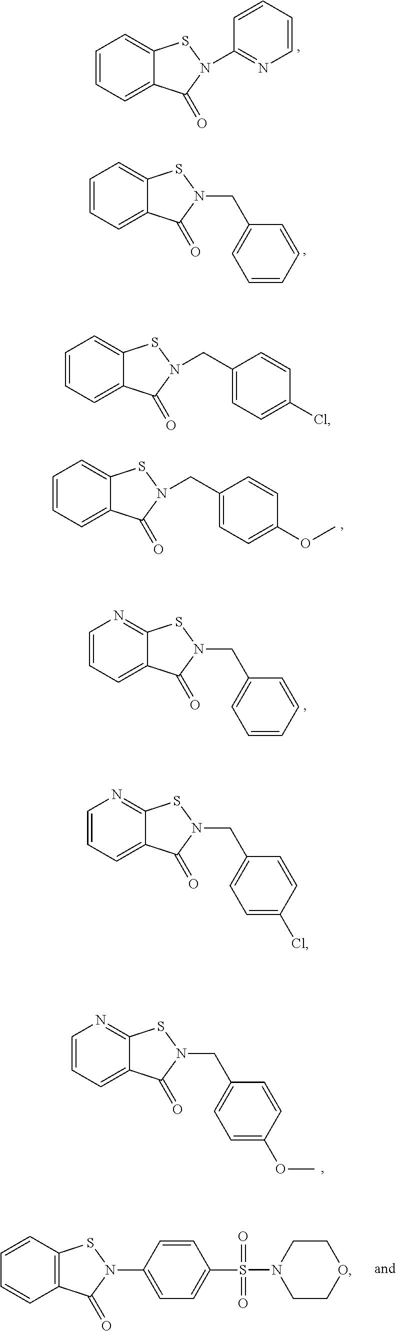

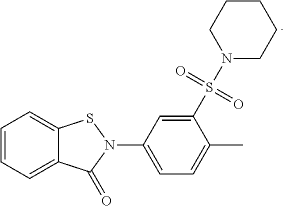 [SMILES: Cc1ccc(-n2sc3ccccc3c2=O)cc1S(=O)(=O)N1CCCCC1]

In another aspect, the IspD inhibitors of the present invention include compounds of Formula II

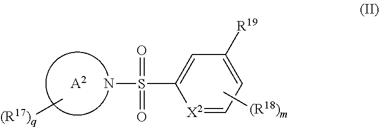

where

X² is C—R¹⁶, or N;

R¹⁶ is hydrogen, halo, hydroxy, C₁-C₄ alkyl, C₁-C₄ alkoxy, halo-substituted C₁-C₄ alkyl, or amino;

each R¹⁷ is independently halo, hydroxy, C₁-C₄ alkyl, C₁-C₄ alkoxy, halo-substituted C₁-C₄ alkyl, or amino;

each R¹⁸ is independently halo, hydroxy, C₁-C₄ alkyl, C₁-C₄ alkoxy, halo-substituted C₁-C₄ alkyl, or amino;

R¹⁹ is

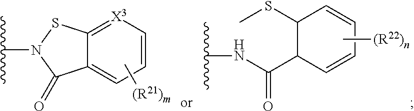

X³ is C—R²⁰ or N;

R²⁰ is hydrogen, halo, hydroxy, C₁-C₄ alkyl, C₁-C₄ alkoxy, halo-substituted C₁-C₄ alkyl, or amino;

each R²¹ is independently halo, hydroxy, C₁-C₄ alkyl, C₁-C₄ alkoxy, halo-substituted C₁-C₄ alkyl, or amino;

each R²² is independently hydrogen, halo, hydroxy, C₁-C₄ alkyl, C₁-C₄ alkoxy, halo-substituted C₁-C₄ alkyl, or amino;

A² is an aliphatic heterocyclic ring;

each m is independently 0 to 3;

n is 0 to 4; and

q is 0 to 10.

In various embodiments, X² is C—R¹⁶ and R¹⁶ is hydrogen or C₁-C₄ alkyl (e.g., methyl or ethyl). In some embodiments A² is a 6 or 7-membered aliphatic heterocyclic ring optionally containing one or more additional heteroatoms selected from the group consisting of oxygen, nitrogen and combinations thereof.

In these and other embodiments, the compounds of Formula II are characterized by one or more of the following:

each m is independently 0 to 1;

n is 0 to 1;

q is 0 to 2 (e.g., 0 to 1);

each R¹⁷ and R¹⁸ are independently halo (e.g., F or Cl), hydroxy, C₁-C₄ alkyl (e.g., methyl or ethyl), C₁-C₄ alkoxy (e.g., methoxy or ethoxy), or amino;

X³ is C—R²⁰ and R²⁰ is hydrogen, hydroxy, C₁-C₄ alkyl, or C₁-C₄ alkoxy; and

R²¹ and R²² are each independently halo (e.g., F or Cl), hydroxy, C₁-C₄ alkyl, C₁-C₄ alkoxy, trifluoromethyl, or amino.

In certain embodiments, the IspD inhibitor of Formula II is selected from the group consisting of:

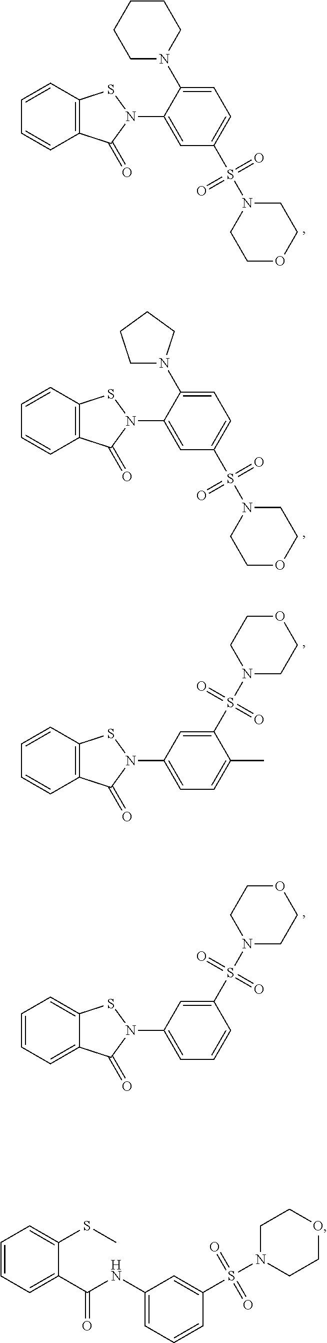 [SMILES: O=c1c2ccccc2sn1-c1cc(S(=O)(=O)N2CCOCC2)ccc1N1CCCCC1 ; O=c1c2ccccc2sn1-c1cc(S(=O)(=O)N2CCOCC2)ccc1N1CCCC1 ; Cc1ccc(-n2sc3ccccc3c2=O)cc1S(=O)(=O)N1CCOCC1 ; O=c1c2ccccc2sn1-c1cccc(S(=O)(=O)N2CCOCC2)c1 ; CSc1ccccc1C(=O)Nc1cccc(S(=O)(=O)N2CCOCC2)c1]

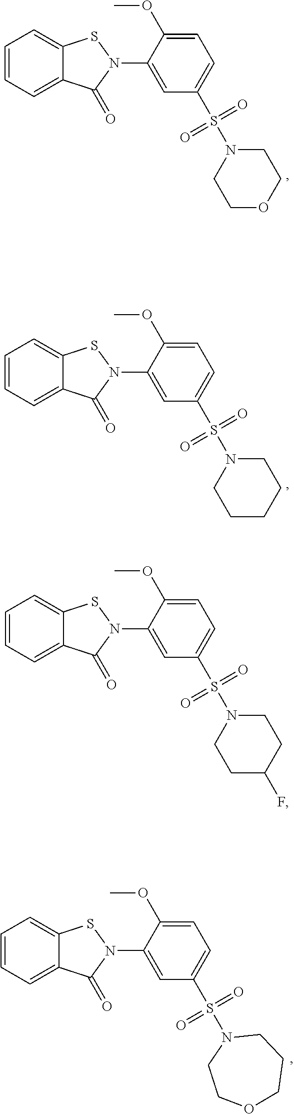 [SMILES: COc1ccc(S(=O)(=O)N2CCOCC2)cc1-n1sc2ccccc2c1=O ; COc1ccc(S(=O)(=O)N2CCCCC2)cc1-n1sc2ccccc2c1=O ; COc1ccc(S(=O)(=O)N2CCC(F)CC2)cc1-n1sc2ccccc2c1=O ; COc1ccc(S(=O)(=O)N2CCCOCC2)cc1-n1sc2ccccc2c1=O]

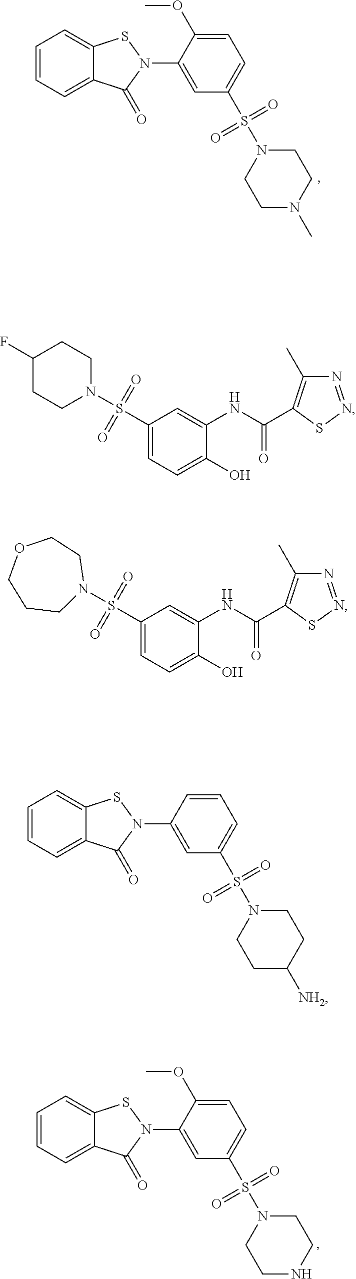 [SMILES: COc1ccc(S(=O)(=O)N2CCN(C)CC2)cc1-n1sc2ccccc2c1=O ; Cc1nnsc1C(=O)Nc1cc(S(=O)(=O)N2CCC(F)CC2)ccc1O ; Cc1nnsc1C(=O)Nc1cc(S(=O)(=O)N2CCCOCC2)ccc1O ; NC1CCN(S(=O)(=O)c2cccc(-n3sc4ccccc4c3=O)c2)CC1 ; COc1ccc(S(=O)(=O)N2CCNCC2)cc1-n1sc2ccccc2c1=O]

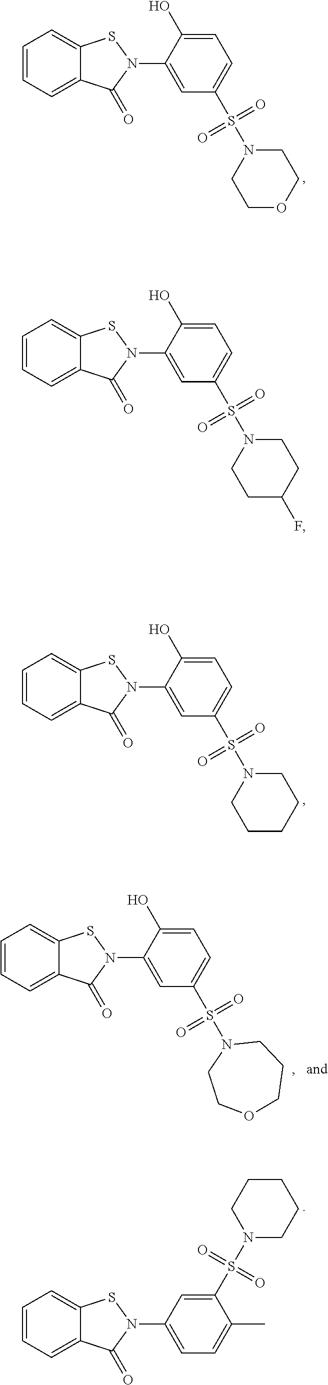 [SMILES: O=c1c2ccccc2sn1-c1cc(S(=O)(=O)N2CCOCC2)ccc1O ; O=c1c2ccccc2sn1-c1cc(S(=O)(=O)N2CCC(F)CC2)ccc1O ; O=c1c2ccccc2sn1-c1cc(S(=O)(=O)N2CCCCC2)ccc1O ; O=c1c2ccccc2sn1-c1cc(S(=O)(=O)N2CCCOCC2)ccc1O ; Cc1ccc(-n2sc3ccccc3c2=O)cc1S(=O)(=O)N1CCCCC1]

In addition to the IspD inhibitor compounds of Formulas I and II, it has been discovered that other compounds inhibit the IspD enzyme. Additional IspD inhibitors include the following compounds:

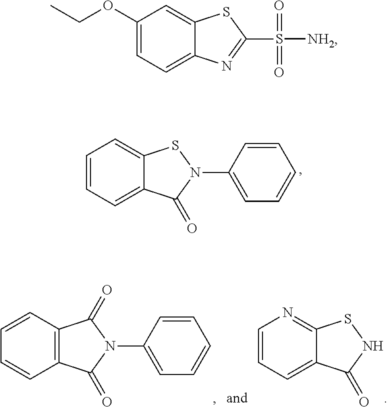 [SMILES: CCOc1ccc2nc(S(N)(=O)=O)sc2c1 ; O=c1c2ccccc2sn1-c1ccccc1 ; O=C1c2ccccc2C(=O)N1c1ccccc1 ; O=c1[nH]sc2ncccc12]

The present invention is also directed to methods of treating diseases or conditions caused by organisms possessing the MEP pathway, particularly organisms where isoprenoid biosynthesis via the MEP pathway has been shown to be essential to development. For example, the IspD inhibitors of the present invention are capable of killing disease-causing parasites possessing the MEP pathway such as P. falciparum and P. vivax. In various embodiments, the methods of the present invention include treating malaria that is caused by P. falciparum and related organisms.

In general, the methods of treating diseases such as malaria comprises administering to a subject in need thereof a pharmaceutical composition comprising a therapeutically effective amount of at least one IspD inhibitor (i.e., a compound of Formulas I, II, the additional IspD inhibitors mentioned herein, and combinations thereof). Typically, the subject is a mammal and more particularly a human subject infected with the disease.

In accordance with other aspects of the present invention, the IspD compounds of present invention may be formulated in a suitable pharmaceutical composition. Generally, the pharmaceutical composition comprises a therapeutically effective amount of at least one IspD inhibitor (i.e., a compound of Formulas I, II, the additional IspD inhibitors mentioned herein, and combinations thereof) and one or more excipients.

The pharmaceutical compositions containing the compounds of the present invention may be formulated in any conventional manner. Proper formulation is dependent in part upon the route of administration selected. Routes of administration include, but are not limited to, oral, parenteral, topical, and so on.

Pharmaceutically acceptable excipients for use in the compositions of the present invention are selected based upon a number of factors including the particular compound used, and its concentration, stability and intended bioavailability; the disease, disorder or condition being treated with the composition; the subject, its age, size and general condition; and the route of administration.

In various embodiments, the pharmaceutical composition comprises an oral vehicle comprising an IspD inhibitor compound. The pharmaceutical compositions can be formulated as tablets, dispersible powders, pills, capsules, gel-caps, granules, solutions, suspensions, emulsions, syrups, elixirs, troches, lozenges, or any other dosage form that can be administered orally. Pharmaceutical compositions for oral administration may include one or more pharmaceutically acceptable excipients. Suitable excipients for solid dosage forms include sugars, starches, and other conventional substances including lactose, talc, sucrose, gelatin, carboxymethylcellulose, agar, mannitol, sorbitol, calcium phosphate, calcium carbonate, sodium carbonate, kaolin, alginic acid, acacia, corn starch, potato starch, sodium saccharin, magnesium carbonate, microcrystalline cellulose, colloidal silicon dioxide, croscarmellose sodium, talc, magnesium stearate, and stearic acid. Further, such solid dosage forms may be uncoated or may be coated to delay disintegration and absorption.

In various embodiments, the pharmaceutical compositions of the present invention is formulated for parenteral administration, e.g., formulated for injection via intravenous, intraarterial, subcutaneous, rectal, subcutaneous, intramuscular, intraorbital, intracapsular, intraspinal, intraperitoneal, or intrasternal routes. Dosage forms suitable for parenteral administration include solutions, suspensions, dispersions, emulsions or any other dosage form that can be administered parenterally.

Pharmaceutically acceptable excipients are identified, for example, in The Handbook of Pharmaceutical Excipients, (American Pharmaceutical Association, Washington, D.C., and The Pharmaceutical Society of Great Britain, London, England, 1968) Additional excipients can be included in the pharmaceutical compositions of the invention for a variety of purposes. These excipients may impart properties which enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, non-volatile silicones, adhesives, bulking agents, flavorings, sweeteners, adsorbents, binders, disintegrating agents, lubricants, coating agents, and antioxidants.

Having described the invention in detail, it will be apparent that modifications and variations are possible without departing from the scope of the invention defined in the appended claims.

### EXAMPLES

The following non-limiting examples are provided to further illustrate the present invention.

### Example 1

Inhibition of Malaria IspD Enzyme

Compounds listed in Table 1 were tested for activity against purified recombinant Plasmodium falciparum methylerythritol cytidyltransferase (PfIspD) enzyme. Reactions were continuously monitored for phosphate release, as previously described for bacterial IspD in Zhang et al., Biochemistry, (2011) 50(17), 3570-3577, which is incorporated herein by reference. Each compound was evaluated for enzymatic inhibition over several concentrations. Nonlinear regression analysis (GraphPad Prism) was used to determine the half-maximal inhibitory concentration (IC₅₀) for each compound.

Antimalarial Efficacy

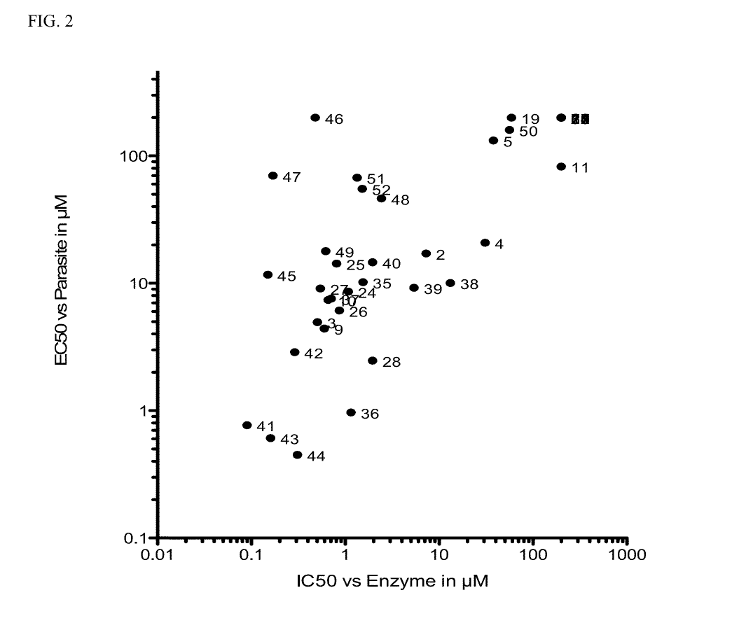

Each compound was tested for the ability to inhibit growth of a standard cultured Plasmodium falciparum parasite strain 3D7. Parasite growth was monitored by staining with the DNA fluorophore Sybr Green, as previously described for the antimalarial fosmidomycin in Zhang et al., Biochemistry, (2011) 50(17), 3570-3577. Each compound was evaluated for antimalarial activity over several concentrations. Nonlinear regression analysis was used to determine the concentration of half-maximal antimalarial efficacy (EC₅₀) for each compound. Comparison of activity against the target enzyme (IC50) and antimalarial efficacy (EC5) for each compound demonstrated that increased potency against the target was highly correlated with increased efficacy against malaria parasites. See FIG. 2. These data show that the antimalarial efficacy of the tested compounds is through cellular inhibition of the target ISPD enzyme.

TABLE 1

| | | IC50 | EC50 |
|---|---|---|---|
| | | (vs enzyme) in | (vs parasite) |
| Compound # | Chemical Name | μM | in μM |
| 2 | 1,2-benzisothiazol-3(2H)-one,2-[-5-(4-morpholinyl | 7.26196 | 17.14 |
| | sulfonyl)-2-(1-pyrrolidinyl)phenyl] | | |
| 3 | 1,2-benzisothiazol-3(2H)-one,2-[4-methyl-3-(4- | 0.5063066 | 4.96 |
| | morpholinyl sulfonyl)phenyl] | | |
| 4 | 1,2-benzisothiazol-3(2H)-one,2-[4-(4-morpholinyl | 30.8861 | 20.82 |
| | sulfonyl)phenyl] | | |
| 9 | 2-phenylbenzo[d]isothiazol-3(2H)-one | 0.5995 | 4.42 |
| 10 | 2-(3-morpholinosulfonyl)phenylbenzo[d]isothiazol-3(2H)- | 0.6556 | 7.40 |
| | one | | |
| 11 | 2-(methylthio)-(N-((3- | >200 | 82.62 |
| | morpholinosulfonyl)phenyl)benzamide) | | |
| 19 | N-[2-hydroxy-5-(piperidine-1-sulfonyl)phenyl]-4-methyl- | 58.86 | >200 |
| | 1,2,3-thiadiazole-5-carboxamide | | |
| 24 | 2-(2-methoxy-5- | 1.077 | 8.61 |
| | (morpholinosulfonyl)phenyl)benzo[d]isothiazol-3(2H)-one | | |
| 25 | 2-(2-methoxy-5-(piperidin-1- | 0.8091 | 14.30 |
| | ylsulfonyl)phenyl)benzo[d]isothiazol-3(2H)-one | | |
| 26 | 2-(5-((4-fluoropiperidin-1-yl)sulfonyl)-2- | 0.8662 | 6.11 |
| | methoxyphenyl)benzo[d]isothiazol-3(2H)-one | | |
| 27 | 2-(5-((1,4-oxazepan-4-yl)sulfonyl)-2- | 0.5424 | 9.10 |
| | methoxyphenyl)benzo[d]isothiazol-3(2H)-one | | |
| 28 | 2-(2-methoxy-5-((4-methylpiperazin-1- | 1.954 | 2.47 |
| | yl)sulfonyl)phenyl)benzo[d]isothiazol-3(2H)-one | | |
| 35 | 2-(5-((4-aminopiperidin-1- | 1.55 | 10.20 |
| | yl)sulfonyl)phenyl)benzo[d]isothiazol-3(2H)-one | | |
| 36 | 2-(2-methoxy-5-(piperazin-1- | 1.15 | 0.97 |
| | ylsulfonyl)phenyl)benzo[d]isothiazol-3(2H)-one | | |
| 37 | 2-(2-hydroxy-5- | 0.71 | 7.59 |
| | (morpholinosulfonyl)phenyl)benzo[d]isothiazol-3(2H)-one | | |
| 38 | 2-(5-((4-fluoropiperidin-1-yl)sulfonyl)-2- | 13.15 | 10.06 |
| | hydroxyphenyl)benzo[d]isothiazol-3(2H)-one | | |
| 39 | 2-(2-hydroxy-5-(piperidin-1- | 5.39 | 9.21 |
| | ylsulfonyl)phenyl)benzo[d]isothiazol-3(2H)-one | | |
| 40 | 2-(5-((1,4-oxazepan-4-yl)sulfonyl)-2- | 1.95 | 14.63 |
| | hydroxyphenyl)benzo[d]isothiazol-3(2H)-one | | |
| 41 | 2-(4′-methoxy-[1,1′biphenyl]-3-yl)benzo[d]isothiazol- | 0.09 | 0.77 |
| | 3(2H)-one | | |
| 42 | 2-(3-(thiophen-3-yl)phenyl)benzo[d]isothiazol-3(2H)-one | 0.29 | 2.88 |
| 43 | 2-(4′-chloro-[1,1′biphenyl]-3-yl)benzo[d]isothiazol-3(2H)- | 0.16 | 0.61 |
| | one | | |
| 44 | 2-(4′-(trifluoromethyl)-[1,1′biphenyl]-3- | 0.31 | 0.45 |
| | yl)benzo[d]isothiazol-3(2H)-one | | |
| 45 | Methyl-3-(3-oxobenzo[d]isothiazol-2(3H)-yl)benzoate | 0.15 | 11.70 |
| 46 | 3-(3-oxobenzo[d]isothiazol-2(3H)-yl)benzoic acid | 0.48 | >200 |
| 47 | 2-(pyridin-2-yl)benzo[d]isothiazol-2(3H)-one | 0.17 | 70.02 |
| 48 | 2-benzylbenzo[d]isothiazol-2(3H)-one | 2.43 | 46.45 |
| 49 | 2-(4-chlorobenzyl)benzo[d]isothiazol-2(3H)-one | 0.62 | 17.88 |
| 50 | Isothiazolo[5,4-b]pyridin-3(2H)-one | 56.17 | 160.16 |
| 51 | 2-(4-methoxybenzyl)benzo[d]isothiazol-2(3H)-one | 1.34 | 67.60 |
| 52 | 2-benzylisothiazolo[5,4-b]pyridin-3(2H)-one | 1.52 | 55.26 |

Mechanism of Action

Metabolic Profiling of Inhibitor-Treated Malaria Parasites

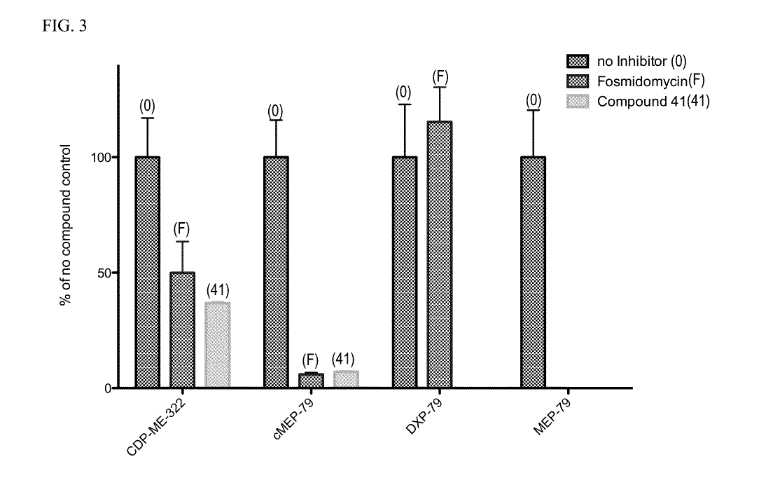

A quantitative liquid chromatography-mass spectrometry method (LC-MS/MS) was developed to determine the cellular levels of metabolites in the MEP pathway. This method allows determination of whether the MEP pathway has been inhibited in malaria parasites treated with MEP pathway inhibitors of the present invention. This method has been previously used to validate the cellular metabolic effects of the known MEP pathway inhibitor fosmidomycin (Zhang et al., Biochemistry 2011). This method was used to determine whether the compounds of the present invention also inhibited the MEP pathway. It was found that downstream metabolites in the MEP pathway (CDP-ME and cMEPP) are significantly decreased when parasite lines are treated with compound 41 (2-(4′-methoxy-[1,1′biphenyl]-3-yl)benzo[d]isothiazol-3(2H)-one), a representative IspD inhibitor of the present invention. FIG. 3 presents these results.

### REFERENCES

1. M. A. Phillips, P. Leon, A. Boronat and M. Rodriguez-Concepcion, “The plastidial MEP pathway: unified nomenclature and resources,” Trends Plant Sci., 2008, 13(12), 619-623.

2. H. Jomaa, J. Wiesner, S. Sanderbrand, B. Altincicek, C. Weidemeyer, M. Hintz, I. Türbachova, M. Eberl, J. Zeidler, H. K. Lichtenthaler, D. Soldati and E. Beck, “Inhibitors of the Nonmevalonate Pathway of Isoprenoid Biosynthesis as Antimalarial Drugs,” Science, 1999, 285(5433), 1573-1576.

3. A. Brown and T. Parish, “Dxr is essential in Mycobacterium tuberculosis and fosmidomycin resistance is due to a lack of uptake,” BMC Microbiol., 2008, 8(1), 78.

4. R. M. Cornish, J. R. Roth and C. D. Poulter, “Lethal Mutations in the Isoprenoid Pathway of Salmonella enterica,” J. Bacteriol., 2006, 188(4), 1444-1450.

5. T. Kuzuyama, S. Takahashi and H. Seto, “Construction and characterization of Escherichia coli disruptants defective in the yaeM gene,” Biosci., Biotechnol., Biochem., 1999, 63(4), 776-8.

6. S. C. Nair, C. F. Brooks, C. D. Goodman, A. Strurm, G. I. McFadden, S. Sundriyal, J. L. Anglin, Y. Song, S. N. J. Moreno and B. Striepen, “Apicoplast isoprenoid precursor synthesis and the molecular basis of fosmidomycin resistance in Toxoplasma gondii,” J. Exp. Med., 2011, 208(7), 1547-1559.

7. A. R. Odom and W. C. Van Voorhis, “Functional genetic analysis of the Plasmodium falciparum deoxyxylulose 5-phosphate reductoisomerase gene, Mol. Biochem. Parasitol., 2010, 170(2), 108-11.

8. A. C. Brown, M. Eberl, D. C. Crick, H. Jomaa and T. Parish, “The Nonmevalonate Pathway of Isoprenoid Biosynthesis in Mycobacterium tuberculosis is Essential and Transcriptionally Regulated by Dxs,” J. Bacteriol., 2010, 192(9), 2424-2433.

9. E. Yeh and J. L. DeRisi, “Chemical Rescue of Malaria Parasites Lacking an Apicoplast Defines Organelle Function in Blood-Stage,” PLoS Biol., 2011, 9(8), e1001138.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and methods without departing from the scope of the invention, it is intended that all matter contained in the above description and shown in the accompanying figures shall be interpreted as illustrative and not in a limiting sense.

1. A compound of Formula I 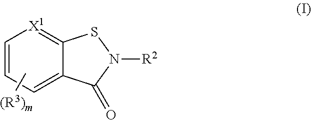
where
X¹ is C—R¹ or N;
R¹ is hydrogen, halo, hydroxy, C₁-C₄ alkyl, C₁-C₄ alkoxy, halo-substituted C₁-C₄ alkyl, or amino;
R² is: 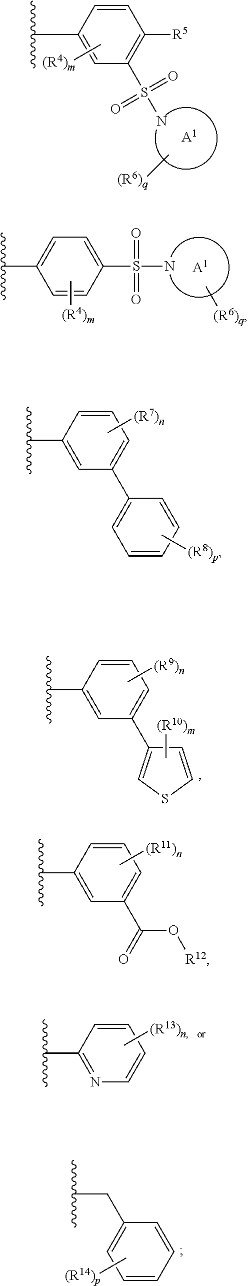
each R³ is independently halo, hydroxy, C₁-C₄ alkyl, C₁-C₄ alkoxy, halo-substituted C₁-C₄ alkyl, or amino;
each R⁴ is halo, hydroxy, C₁-C₄ alkyl, C₁-C₄ alkoxy, halo-substituted C₁-C₄ alkyl, amino, or a nitrogen-containing aliphatic ring;
R⁵ is hydrogen, halo, hydroxy, C₁-C₄ alkyl, C₁-C₄ alkoxy, halo-substituted C₁-C₄ alkyl, or amino;
each R⁶ is independently halo, hydroxy, C₁-C₄ alkyl, C₁-C₄ alkoxy, halo-substituted C₁-C₄ alkyl, or amino;
each R⁷ is independently halo, hydroxy, C₁-C₄ alkyl, C₁-C₄ alkoxy, halo-substituted C₁-C₄ alkyl, or amino;
each R⁸ is independently halo, hydroxy, C₁-C₄ alkyl, C₁-C₄ alkoxy, halo-substituted C₁-C₄ alkyl, or amino;
each R⁹ is independently halo, hydroxy, C₁-C₄ alkyl, C₁-C₄ alkoxy, halo-substituted C₁-C₄ alkyl, or amino;
each R¹⁰ is independently halo, hydroxy, C₁-C₄ alkyl, C₁-C₄ alkoxy, halo-substituted C₁-C₄ alkyl, or amino;
each R¹¹ is halo, hydroxy, C₁-C₄ alkyl, C₁-C₄ alkoxy, halo-substituted C₁-C₄ alkyl, or amino;
R¹² is hydrogen or C₁-C₄ alkyl;
each R¹³ is independently halo, hydroxy, C₁-C₄ alkyl, C₁-C₄ alkoxy, halo-substituted C₁-C₄ alkyl, or amino;
each R¹⁴ is independently halo, hydroxy, C₁-C₄ alkyl, C₁-C₄ alkoxy, halo-substituted C₁-C₄ alkyl, or amino;
each A¹ is an aliphatic heterocyclic ring;
each m is independently 0 to 3;
each n is independently 0 to 4;
each p is independently 0 to 5; and
q is 0 to 10.

2. The compound of claim 1 wherein X¹ is C—R¹ and R¹ is hydrogen, halo, hydroxy, C₁-C₄ alkyl, or C₁-C₄ alkoxy.

3. The compound of claim 1 which is characterized by one or more of the following:
R² is 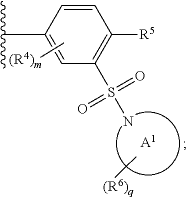
R⁴ is hydroxy, C₁-C₄ alkyl, C₁-C₄ alkoxy, or a 5 to 7-membered nitrogen-containing aliphatic ring;
R⁵ is hydrogen, hydroxy, C₁-C₄ alkyl, or C₁-C₄ alkoxy;
R⁶ is halo, C₁-C₄ alkyl, C₁-C₄ alkoxy, or amino;
A¹ is a 6 or 7-membered aliphatic heterocyclic ring optionally containing one or more additional heteroatoms selected from the group consisting of oxygen, nitrogen and combinations thereof;
m is 0 to 1; and
q is 0 to 1.

4. The compound of claim 1 wherein the compound of Formula I is selected from the group consisting of:

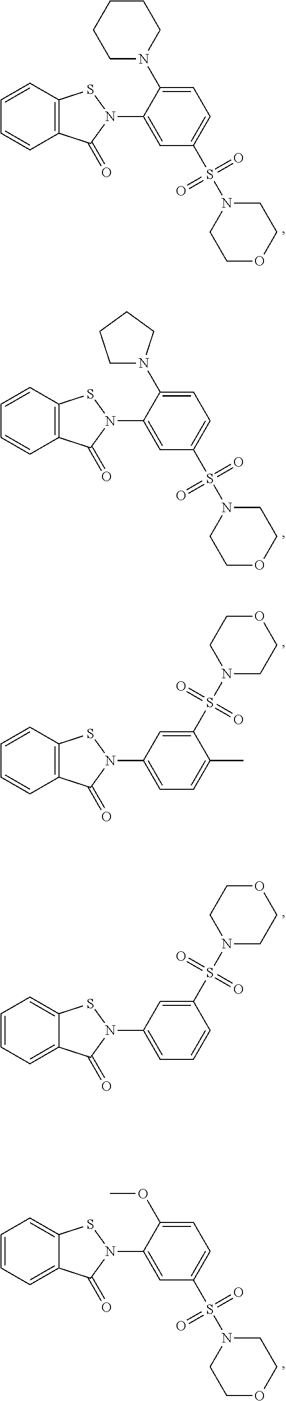 [SMILES: O=c1c2ccccc2sn1-c1cc(S(=O)(=O)N2CCOCC2)ccc1N1CCCCC1 ; O=c1c2ccccc2sn1-c1cc(S(=O)(=O)N2CCOCC2)ccc1N1CCCC1 ; Cc1ccc(-n2sc3ccccc3c2=O)cc1S(=O)(=O)N1CCOCC1 ; O=c1c2ccccc2sn1-c1cccc(S(=O)(=O)N2CCOCC2)c1 ; COc1ccc(S(=O)(=O)N2CCOCC2)cc1-n1sc2ccccc2c1=O]

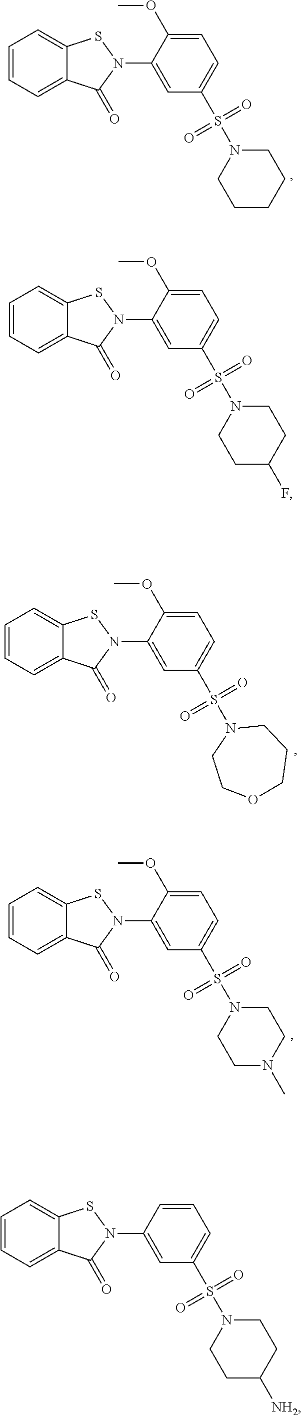 [SMILES: COc1ccc(S(=O)(=O)N2CCCCC2)cc1-n1sc2ccccc2c1=O ; COc1ccc(S(=O)(=O)N2CCC(F)CC2)cc1-n1sc2ccccc2c1=O ; COc1ccc(S(=O)(=O)N2CCCOCC2)cc1-n1sc2ccccc2c1=O ; COc1ccc(S(=O)(=O)N2CCN(C)CC2)cc1-n1sc2ccccc2c1=O ; NC1CCN(S(=O)(=O)c2cccc(-n3sc4ccccc4c3=O)c2)CC1]

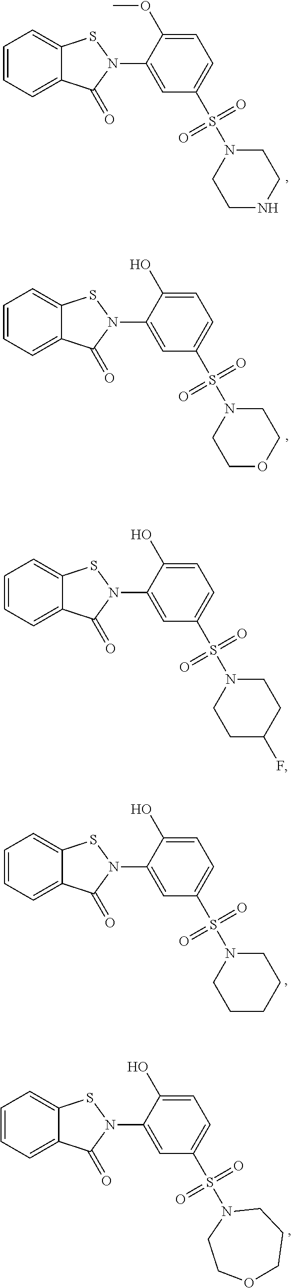 [SMILES: COc1ccc(S(=O)(=O)N2CCNCC2)cc1-n1sc2ccccc2c1=O ; O=c1c2ccccc2sn1-c1cc(S(=O)(=O)N2CCOCC2)ccc1O ; O=c1c2ccccc2sn1-c1cc(S(=O)(=O)N2CCC(F)CC2)ccc1O ; O=c1c2ccccc2sn1-c1cc(S(=O)(=O)N2CCCCC2)ccc1O ; O=c1c2ccccc2sn1-c1cc(S(=O)(=O)N2CCCOCC2)ccc1O]

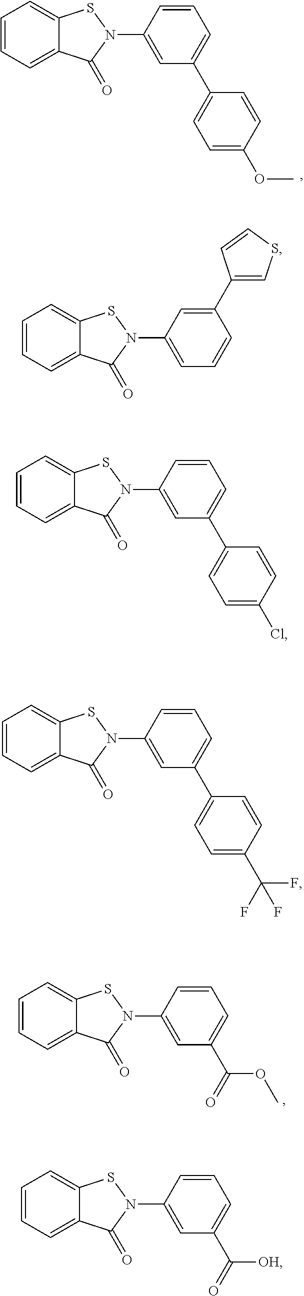 [SMILES: COc1ccc(-c2cccc(-n3sc4ccccc4c3=O)c2)cc1 ; O=c1c2ccccc2sn1-c1cccc(-c2ccsc2)c1 ; O=c1c2ccccc2sn1-c1cccc(-c2ccc(Cl)cc2)c1 ; O=c1c2ccccc2sn1-c1cccc(-c2ccc(C(F)(F)F)cc2)c1 ; COC(=O)c1cccc(-n2sc3ccccc3c2=O)c1 ; O=C(O)c1cccc(-n2sc3ccccc3c2=O)c1]

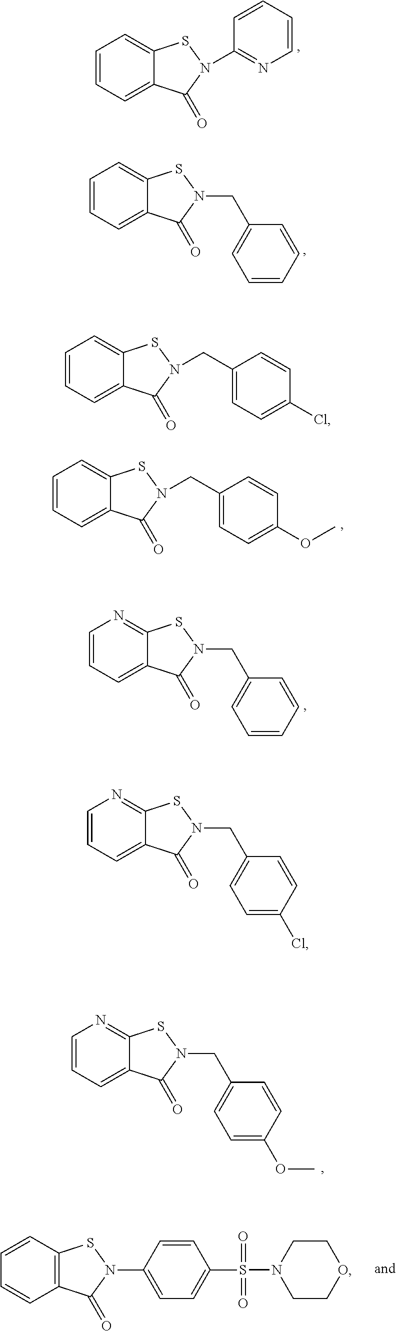

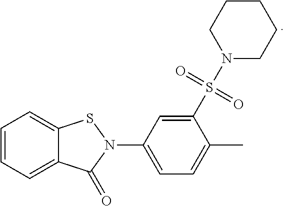 [SMILES: Cc1ccc(-n2sc3ccccc3c2=O)cc1S(=O)(=O)N1CCCCC1]

5. A compound of Formula II 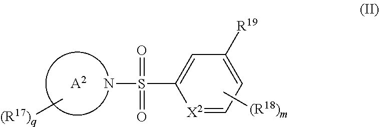
where
X² is C—R¹⁶, or N;
R¹⁶ is hydrogen, halo, hydroxy, C₁-C₄ alkyl, C₁-C₄ alkoxy, halo-substituted C₁-C₄ alkyl, or amino;
each R¹⁷ is independently halo, hydroxy, C₁-C₄ alkyl, C₁-C₄ alkoxy, halo-substituted C₁-C₄ alkyl, or amino;
each R¹⁸ is independently halo, hydroxy, C₁-C₄ alkyl, C₁-C₄ alkoxy, halo-substituted C₁-C₄ alkyl, or amino;
R¹⁹ is 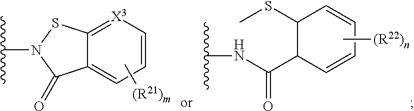
X³ is C—R²⁰ or N;
R²⁰ is hydrogen, halo, hydroxy, C₁-C₄ alkyl, C₁-C₄ alkoxy, halo-substituted C₁-C₄ alkyl, or amino;
each R²¹ is independently halo, hydroxy, C₁-C₄ alkyl, C₁-C₄ alkoxy, halo-substituted C₁-C₄ alkyl, or amino;
each R²² is independently halo, hydroxy, C₁-C₄ alkyl, C₁-C₄ alkoxy, halo-substituted C₁-C₄ alkyl, or amino;
A² is an aliphatic heterocyclic ring;
each m is independently 0 to 3;
n is 0 to 4; and
q is 0 to 10.

6. The compound of claim 5 wherein X² is C—R¹⁶; and R¹⁶ is hydrogen or C₁-C₄ alkyl.

7. The compound of claim 5 wherein A² is a 6 or 7-membered aliphatic heterocyclic ring optionally containing one or more additional heteroatoms selected from the group consisting of oxygen, nitrogen and combinations thereof.

8. The compound of claim 5 wherein the compound of Formula II is characterized by one or more of the following:
each m is independently 0 to 1;
n is 0 to 1;
q is 0 to 2;
each R¹⁷ and R¹⁸ are independently halo, hydroxy, C₁-C₄ alkyl, C₁-C₄ alkoxy, or amino;
X³ is C—R²⁰ and R²⁰ is hydrogen, hydroxy, C₁-C₄ alkyl, or C₁-C₄ alkoxy; and
R²¹ and R²² are each independently halo, hydroxy, C₁-C₄ alkyl, C₁-C₄ alkoxy, trifluoromethyl, or amino.

9. The compound of claim 5 wherein the compound of Formula II is selected from the group consisting of:

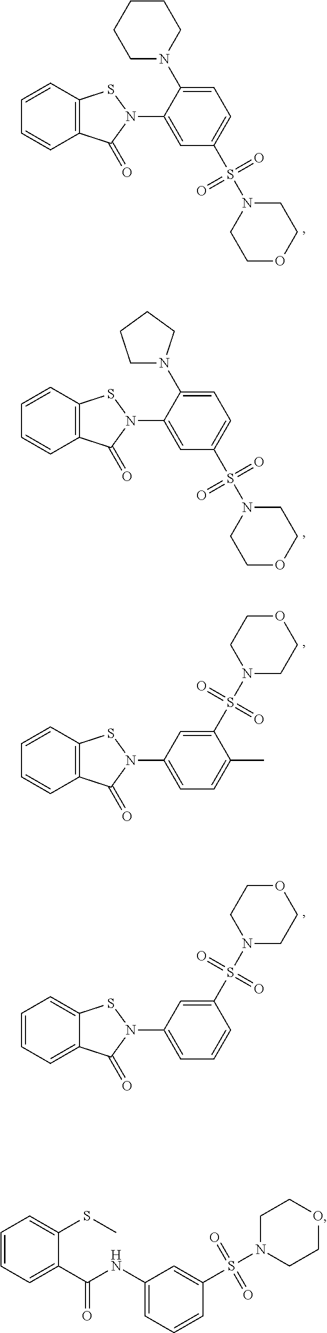 [SMILES: O=c1c2ccccc2sn1-c1cc(S(=O)(=O)N2CCOCC2)ccc1N1CCCCC1 ; O=c1c2ccccc2sn1-c1cc(S(=O)(=O)N2CCOCC2)ccc1N1CCCC1 ; Cc1ccc(-n2sc3ccccc3c2=O)cc1S(=O)(=O)N1CCOCC1 ; O=c1c2ccccc2sn1-c1cccc(S(=O)(=O)N2CCOCC2)c1 ; CSc1ccccc1C(=O)Nc1cccc(S(=O)(=O)N2CCOCC2)c1]

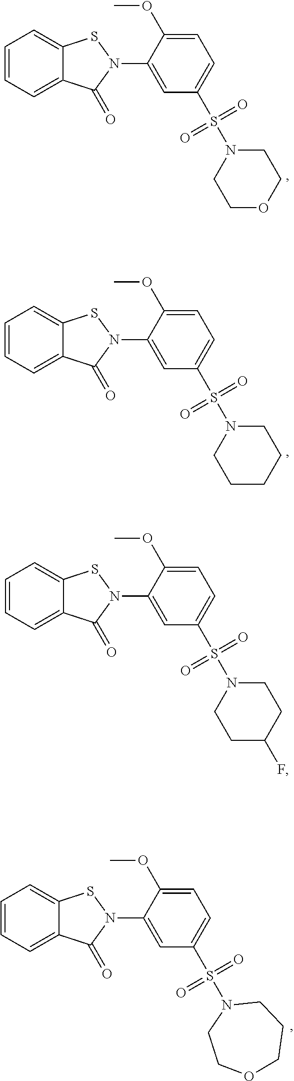 [SMILES: COc1ccc(S(=O)(=O)N2CCOCC2)cc1-n1sc2ccccc2c1=O ; COc1ccc(S(=O)(=O)N2CCCCC2)cc1-n1sc2ccccc2c1=O ; COc1ccc(S(=O)(=O)N2CCC(F)CC2)cc1-n1sc2ccccc2c1=O ; COc1ccc(S(=O)(=O)N2CCCOCC2)cc1-n1sc2ccccc2c1=O]

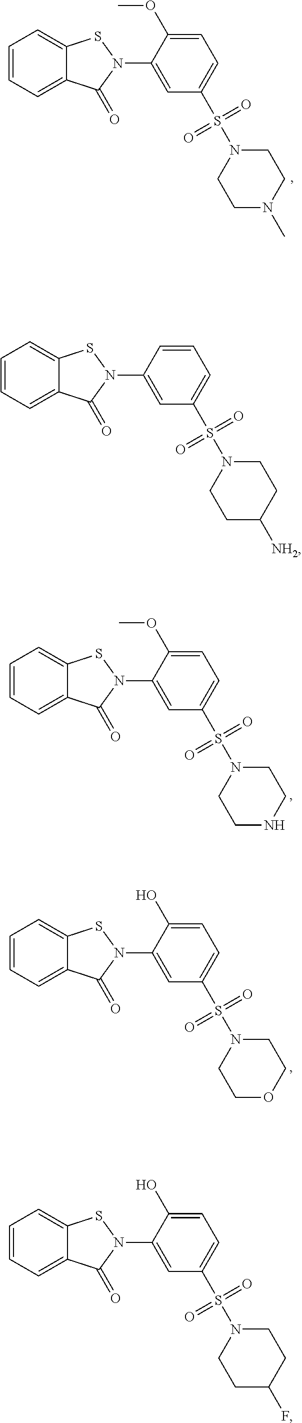 [SMILES: COc1ccc(S(=O)(=O)N2CCN(C)CC2)cc1-n1sc2ccccc2c1=O ; NC1CCN(S(=O)(=O)c2cccc(-n3sc4ccccc4c3=O)c2)CC1 ; COc1ccc(S(=O)(=O)N2CCNCC2)cc1-n1sc2ccccc2c1=O ; O=c1c2ccccc2sn1-c1cc(S(=O)(=O)N2CCOCC2)ccc1O ; O=c1c2ccccc2sn1-c1cc(S(=O)(=O)N2CCC(F)CC2)ccc1O]

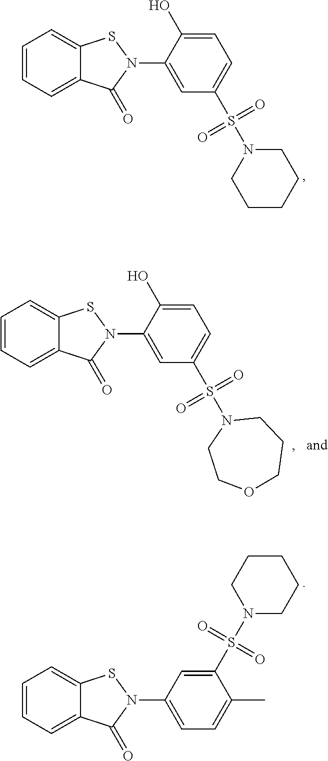 [SMILES: O=c1c2ccccc2sn1-c1cc(S(=O)(=O)N2CCCCC2)ccc1O ; O=c1c2ccccc2sn1-c1cc(S(=O)(=O)N2CCCOCC2)ccc1O ; Cc1ccc(-n2sc3ccccc3c2=O)cc1S(=O)(=O)N1CCCCC1]

10. A pharmaceutical composition comprising the compound of claim 1 and at least one excipient.

11. A method of treating a disease caused by an organism possessing the MEP pathway in a subject in need thereof comprising administering a pharmaceutical composition comprising a therapeutically effective amount of a compound of Formula I as defined in claim 1.

12. A method of treating a disease caused by an organism possessing the MEP pathway in a subject in need thereof comprising administering a pharmaceutical composition comprising a therapeutically effective amount of a compound of selected from the group consisting of

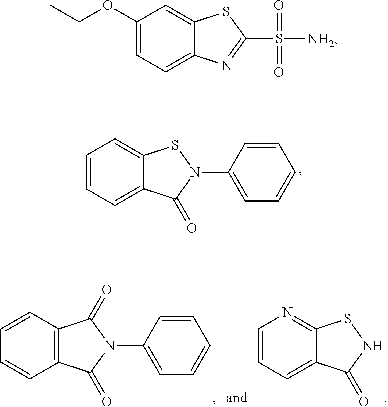 [SMILES: CCOc1ccc2nc(S(N)(=O)=O)sc2c1 ; O=c1c2ccccc2sn1-c1ccccc1 ; O=C1c2ccccc2C(=O)N1c1ccccc1 ; O=c1[nH]sc2ncccc12]

13. The method of claim 12 wherein the disease is malaria.

14. The method of claim 13 wherein the malaria is caused by P. falciparum.

15. A pharmaceutical composition comprising the compound of claim 5 and at least one excipient.

16. A method of treating a disease caused by an organism possessing the MEP pathway in a subject in need thereof comprising administering a pharmaceutical composition comprising a therapeutically effective amount of a compound of Formula II as defined in claim 5.

17. The method of claim 16 wherein the disease is malaria.

18. The method of claim 17 wherein the malaria is caused by P. falciparum.

19. The method of claim 11 wherein the disease is malaria.

20. The method of claim 19 wherein the malaria is caused by P. falciparum.
